(19) United States
(12) Patent Application Publication
(10) Pub. No.: US 2010/0145149 A1
(43) Pub. Date: Jun. 10, 2010

(54) LIVING BODY OBSERVATION SYSTEM AND METHOD OF DRIVING LIVING BODY OBSERVATION SYSTEM

(75) Inventors: Naoki YOSHIDA, Kamiina-gun (JP); Fukashi YOSHIZAWA, Ina-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/624,795

(22) Filed: Nov. 24, 2009

(30) Foreign Application Priority Data
Dec. 1, 2008 (JP) 2008-306774

(51) Int. Cl. A61B 1/00

(52) U.S. Cl. 600/118

(57) ABSTRACT

A living body observation system of the present invention includes an in vivo observation apparatus including: an in vivo information acquisition section; a power supply section; a magnetic field detection section for detecting a magnetic field from outside; and a power supply control section for controlling a supply state of driving power supplied to the in vivo information acquisition section, based on an electric signal, and a magnetic field generation section which generates the magnetic field, the magnetic field generation section including: a transmission antenna which includes a transmission coil which generates an alternating magnetic field for controlling activation and deactivation of the in vivo observation apparatus and a ferromagnetic material which is arranged in an outer periphery of the transmission coil and decreases a leakage of the alternating magnetic field in surroundings, and transmits the alternating magnetic field; a driver for driving the transmission antenna; and a power supply for supplying power to the driver.

This application claims benefit of Japanese Application No. 2008-306774 filed in Japan on Dec. 1, 2008, the contents of which are incorporated by this reference.

### BACKGROUND OF THE INVENTION

1. Field of the Invention

The present invention relates to a living body observation system which can acquire information in a living body through an in vivo observation apparatus, and a method of driving the living body observation system.

2. Description of the Related Art

Conventionally, endoscopes have been widely used in the medical field and others. Endoscopes in the medical field, in particular, are mainly used for the purpose of observing the inside of a living body. Recently, as a type of the aforementioned endoscopes, a capsule endoscope has been proposed, which is disposed in a body cavity by being swallowed by a subject and which can pick up an image of an object while moving in the body cavity incident to peristaltic movements and wirelessly transmit the picked-up image of the object to the outside as an image pickup signal.

Conventionally, endoscopes have been widely used in the medical field and others. Endoscopes in the medical field, in particular, are mainly used for the purpose of observing the inside of a living body. Recently, as a type of the aforementioned endoscopes, a capsule endoscope has been proposed, which is disposed in a body cavity by being swallowed by a subject and which can pick up an image of an object while moving in the body cavity incident to peristaltic movements and wirelessly transmit the picked-up image of the object to the outside as an image pickup signal.

An apparatus having substantially the same function as that of the capsule endoscope described above is proposed in, for example, Japanese Patent Application Laid-Open Publication No. 2001-224553. FIG. 12 is a circuit diagram to illustrate an ON state or OFF state of the power supply of the image pickup apparatus of the capsule endoscope disclosed in Japanese Patent Application Laid-Open Publication No. 2001-224553.

Japanese Patent Application Laid-Open Publication No. 2001-224553 describes, as shown in FIG. 12, the configuration of a capsule endoscope which utilizes, as a non-contact power supply switch, a reed switch 71, contacts of which are adapted to be open when placed in a static magnetic field.

The capsule endoscope described in Japanese Patent Application Laid-Open Publication No. 2001-224553 is configured to have the above described reed switch 71 such that when the capsule endoscope is stored in a packaging box or storage case equipped with a magnet, the contacts of the reed switch 71 are opened so that the power supply is turned off.

Further, the above described capsule endoscope is configured such that when it is taken out of the packaging box or storage case, the contacts of the reed switch 71 are closed so that the power supply is turned on, that is, power is supplied from a battery 70.

### SUMMARY OF THE INVENTION

A living body observation system in the present invention comprises: an in vivo observation apparatus including: an in vivo information acquisition section for acquiring information in a living body; a power supply section for supplying driving power of the in vivo information acquisition section; a magnetic field detection section for detecting an alternating magnetic field from outside and outputting a detection result as an electric signal; and a power supply control section for controlling a supply state of driving power supplied from the power supply section to the in vivo information acquisition section, based on the electric signal; and a magnetic field generation section which is disposed outside the in vivo observation apparatus and generates the alternating magnetic field, the magnetic field generation section including: a transmission antenna which includes a transmission coil which generates the alternating magnetic field for controlling activation and deactivation of the in vivo observation apparatus and a ferromagnetic material which is arranged in an outer periphery of the transmission coil and decreases a leakage of the alternating magnetic field in surroundings, and transmits the alternating magnetic field; a driver for driving the transmission antenna; and a power supply for supplying power to the driver.

A method of driving the living body observation system in the present invention is a driving method for driving the living body observation system comprising at least: an in vivo observation apparatus including: an in vivo information acquisition section for acquiring information in a living body; a power supply section for supplying driving power of the in vivo information acquisition section; a magnetic field detection section for detecting an alternating magnetic field from outside and outputting a detection result as an electric signal; and a power supply control section for controlling a supply state of driving power supplied from the power supply section to the in vivo information acquisition section, based on the electric signal; and a magnetic field generation section which is disposed outside the in vivo observation apparatus and generates the alternating magnetic field, the magnetic field generation section including: a transmission antenna which includes a transmission coil which generates the alternating magnetic field for controlling activation and deactivation of the in vivo observation apparatus and a ferromagnetic material which is arranged in an outer periphery of the transmission coil and decreases a leakage of the alternating magnetic field in surroundings, and transmits the alternating magnetic field; a driver for driving the transmission antenna; and a power supply for supplying power to the driver, wherein every time the alternating magnetic field from the magnetic field generation section is detected, the in vivo observation apparatus repeatedly gets activated and deactivated.

### BRIEF DESCRIPTION OF THE DRAWINGS

FIG. 1 is a configuration diagram to show a configuration of a whole living body observation system relating to a first embodiment of the living body observation system of the present invention;

FIG. 2 is a block diagram to show an example of the internal configuration of the magnetic field generation section of FIG. 1;

FIG. 3 is a configuration diagram to show the external appearance of the transmission antenna in the magnetic field generation section of FIG. 2;

FIG. 4 is a sectional view taken along the line A-A of FIG. 3;

FIG. 5 is a block diagram to show an example of the internal configuration of the capsule endoscope of FIG. 1;

FIG. 6 is a configuration diagram to show a specific configuration of the reception antenna of FIG. 5;

FIGS. 7A to 7D are timing charts to show an example of the operation state of the capsule endoscope of the present embodiment;

FIG. 8 is a sectional view of a magnetic field generation section of a living body observation system relating to a second embodiment of the living observation system of the present invention;

FIG. 9 is a sectional view of a magnetic field generation section of a living body observation system relating to a third embodiment of the living body observation system of the present invention;

FIG. 10 is a configuration diagram to show a configuration of a magnetic field generation section of a living body observation system relating to a fourth embodiment of the living body observation system of the present invention;

FIG. 11 is a sectional view taken along the line B-B of FIG. 10; and

FIG. 12 is a circuit diagram to illustrate an ON state or OFF state of the power supply of an image pickup apparatus of a conventional capsule endoscope.

### DETAILED DESCRIPTION OF THE PREFERRED EMBODIMENTS

Hereafter, embodiments of the present invention will be described with reference to the drawings.

### First Embodiment

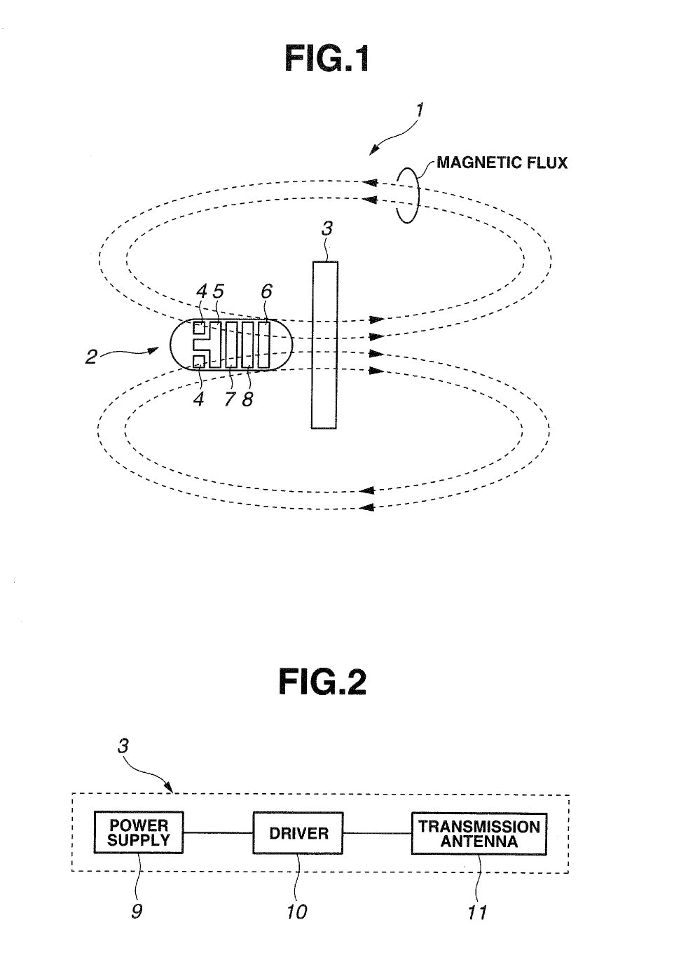

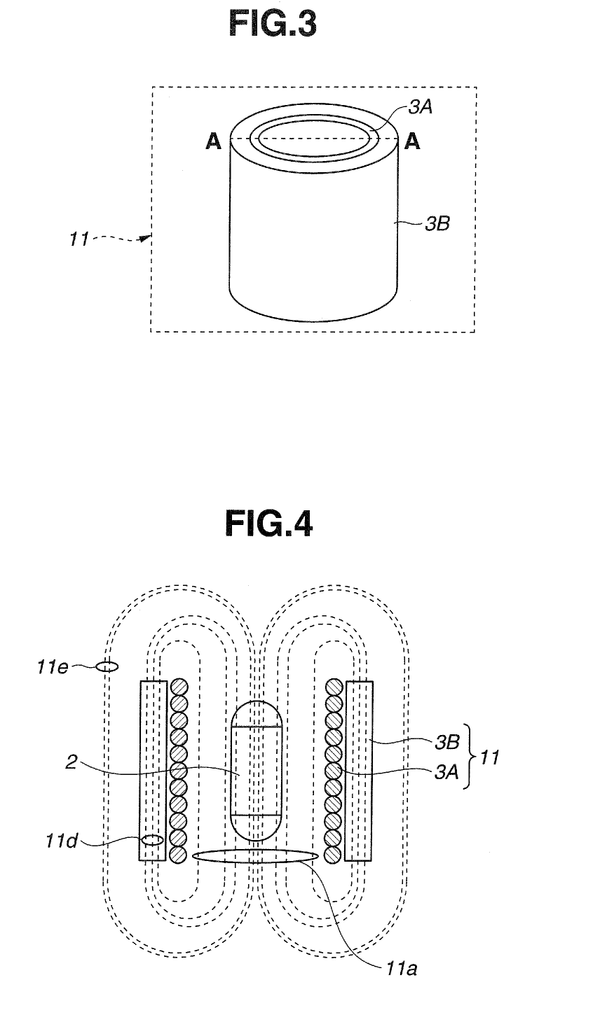

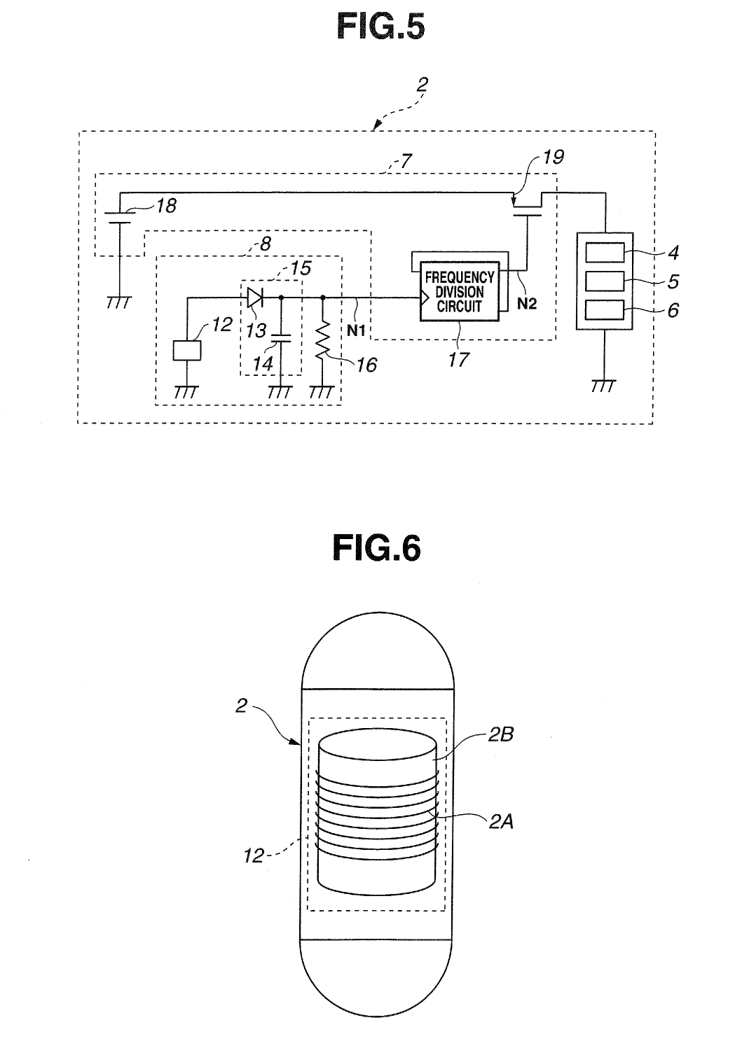

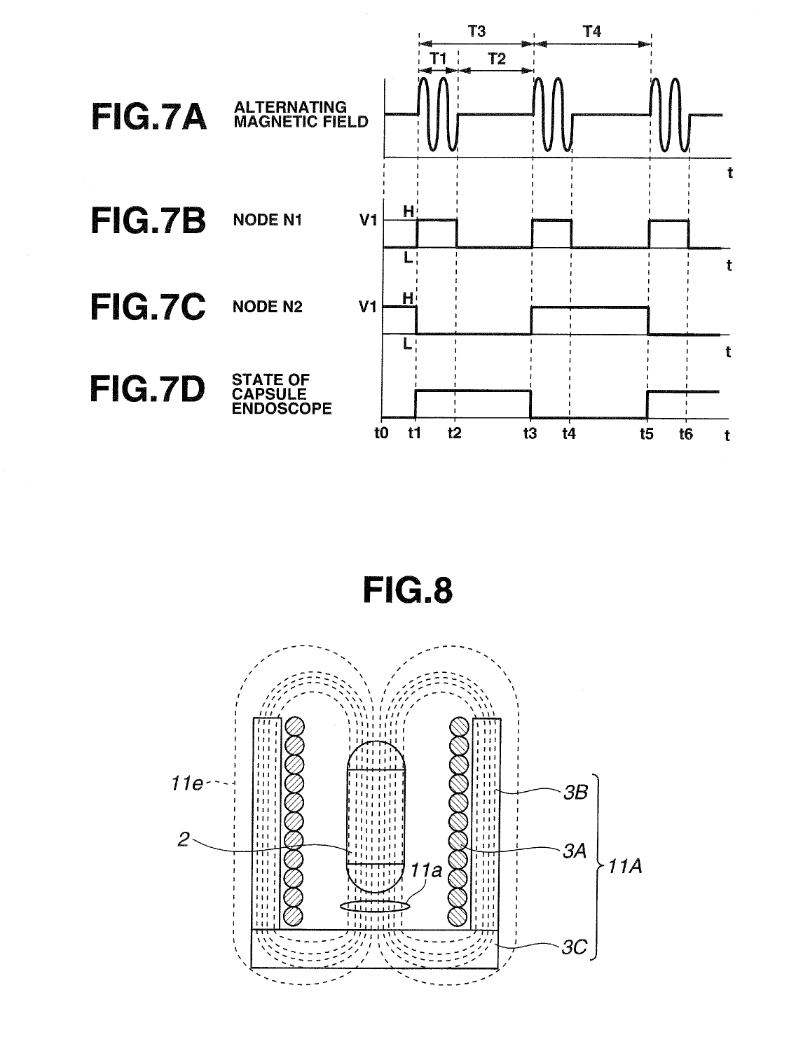

FIG. 1 to FIG. 7D relate to a first embodiment of the living body observation system of the present invention. FIG. 1 is a configuration diagram to show a configuration of the whole living body observation system relating to the first embodiment. FIG. 2 is a block diagram to show an example of the internal configuration of the magnetic field generation section of FIG. 1. FIG. 3 is a configuration diagram to show the external appearance of a transmission antenna in the magnetic field generation section. FIG. 4 is a sectional view taken along the line A-A of FIG. 3. FIG. 5 is a block diagram to show an example of the internal configuration of the capsule endoscope of FIG. 1. FIG. 6 is a configuration diagram to show a specific configuration of a reception antenna of FIG. 5. FIGS. 7A to 7D are timing charts to show an example of the operation state of the capsule endoscope of the present embodiment.

As shown in FIG. 1, a living body observation system 1 of the present embodiment is configured to include a capsule endoscope 2 configured to have an size and shape so as to be able to be disposed in a living body, and a magnetic field generation section 3 which is disposed outside the capsule endoscope 2 and generates an alternating magnetic field.

The magnetic field generation section 3 is configured to be able to turn either on or off the generation state of magnetic field in response to, for example, a user actuating a switch or the like which is not shown. Note that the magnetic field generation section 3 may have any configuration provided that an alternating magnetic field is generated in response to an actuation or direction of the user.

The capsule endoscope 2 as an in vivo observation apparatus incorporates an in vivo information acquisition section which is configured to include at least an illumination section 4 for illuminating an object which is present in front in its own traveling direction, and an image pickup section 5 which has an objective optical system, which is not shown, for forming an image of the object illuminated by the illumination section 4, and outputs the image of the object formed by the objective optical system as an image pickup signal.

Moreover, the capsule endoscope 2 incorporates: a wireless transmission section 6 for transmitting a video signal obtained by the image pickup section 5 to outside the body; a power supply section 7 as a power supply control section for supplying driving power to the illumination section 4, the image pickup section 5, and the wireless transmission section 6 and for controlling the supply of driving power; and a magnetic field detection section 8 for detecting an alternating magnetic field which is externally generated.

Note that an outer housing of the capsule endoscope 2 is configured to have a transparent dome-like lens shape at an end portion in which an image pickup device, which is not shown, is mounted. Further, the remaining cylindrical portion and the opposite end portion of the outer housing are made up of a light shielding material.

In the present embodiment, outside the capsule endoscope 2 having such configuration, a magnetic field generation section 3 for applying an alternating magnetic field to the capsule endoscope 2 is disposed.

Next, specific configurations of the magnetic field generation section 3 shown in FIG. 1 will be described using FIGS. 2 to 4.

As shown in FIG. 2, the magnetic field generation section 3 is configured to include a power supply 9, a driver 10, and a transmission antenna 11.

The power supply 9, which is made up of, for example, a battery etc., supplies power to the driver 10. The driver 10, which is for the purpose of driving the transmission antenna 11, transforms the power supplied from the power supply 9 into a power having a desired frequency and supplies it to the transmission antenna 11 thereby driving the transmission antenna 11.

The transmission antenna 11 generates an alternating magnetic field for controlling the activation and deactivation of the capsule endoscope 2.

Further, the configuration of the transmission antenna 11 will be described using FIGS. 3 and 4.

FIG. 3 shows the external appearance of the transmission antenna 11 shown in FIG. 2.

As shown in FIG. 3, the transmission antenna 11 relating to the present embodiment is configured to include a primary side coil 3A, a yoke 3B disposed in the outer periphery of the primary side coil 3A, and a primary side capacitor which is not shown.

The primary side coil 3A, which makes up a transmission coil, has for example a substantially tubular, solenoid coil shape and is formed such that the capsule endoscope 2 can be inserted into the inside thereof. Further, the yoke 3B is configured to be a cylindrical shape by using, for example, a ferromagnetic material.

Note that the primary side capacitor, which is not shown, makes up a resonance circuit in conjunction with the primary side coil 3A.

Here, the operation of the magnetic field generation section 3 including the transmission antenna 11 having the above described configuration will be described using FIG. 4.

Suppose, for example, as shown in FIG. 4, a user has inserted the capsule endoscope 2 into the inside of the transmission antenna 11. Further suppose that the user has performed the operation to turn on a switch or the like, which is not shown, of the magnetic field generation section 3.

Then, the driver 10 of the magnetic field generation section 3 drives the transmission antenna 11 so that an alternating magnetic field is generated from the primary side coil 3A of the transmission antenna 11.

In this case, as shown in FIG. 4, a magnetic flux in coil 11a generated from the primary side coil 3A is divided into a magnetic flux in yoke 11d which passes through the inside of the yoke 3B, and a leakage magnetic flux 11e which passes through outside the yoke 3B, in the outside of the primary side coil 3A.

In the present embodiment, the magnetic flux of the alternating magnetic filed generated from the transmission antenna 11 is concentrated to the yoke 3B. Therefore, a major portion of the magnetic flux of the alternating magnetic field emitted from the transmission antenna 11 makes up the magnetic flux in yoke 11d, while the leakage magnetic flux 11e becomes very scarce.

The leakage magnetic flux 11e may cause malfunctions of electronic equipment in the surroundings. In the present embodiment, however, since the leakage magnetic flux 11e is very scarce, it becomes possible, as a result, to prevent the electronic equipment in the surroundings from malfunctioning. That is, it becomes possible to dispose other electronic equipment, which is necessary during examination, in the proximity of the magnetic field generation section 3, thereby enabling to improve the diagnostic performance.

Note that the primary side coil 3A making up the transmission antenna 11 is not limited to have a substantially tubular, solenoid coil shape, and may have other shapes.

Next, specific configurations of the power supply section 7 and the magnetic field detection section 8 of the capsule endoscope 2 will be described using FIG. 5.

As shown in FIG. 5, the magnetic field detection section 8 is configured to include a reception antenna 12 for outputting an electric signal in accordance with the alternating magnetic field generated in the magnetic field generation section 3, a rectification section 15 for rectifying and outputting the electric signal outputted from the reception antenna 12, and a resistor 16.

The reception antenna 12 is, although not shown, configured to include, for example, a secondary side coil which is a magnetic field detection coil for outputting an electric signal in accordance with the alternating magnetic field generated at the magnetic field generation section 3, and a resonance capacitor connected in parallel to the magnetic field detection coil (secondary side coil) at the input terminal of the rectification section 15.

The rectification section 15 includes a diode 13, input terminal of which is connected to an output terminal of the reception antenna 12, and a smoothening capacitor 14 for smoothening the electric signal outputted from the diode 13.

The resistor 16 is connected at the output terminal of the diode 13 in parallel with the smoothening capacitor 14.

On the other hand, the power supply section 7 is configured, as shown in FIG. 5, to include a power supply section 18 made up of a battery, etc., a P-channel type FET 19, and a frequency division circuit 17 for dividing into halves the frequency of the output signal (detection signal) from the magnetic field detection section 8.

A node N1 as the input terminal of the frequency division circuit 17 is connected to the output terminal of the magnetic field detection section 8. That is, the electric signal outputted from the magnetic field detection section 8 is inputted into the frequency division circuit 17 via the node N1. A node N2 as the output terminal of the frequency division circuit 17 is connected to a gate of the P-channel type FET 19.

The source of the P-channel type FET 19 is connected to the power supply section 18. Moreover, the gate of the P-channel type FET 19 is connected to the node N2 as the output terminal of the frequency division circuit 17. Further, a drain of the P-channel type FET 19 is connected to an illumination section 4, an image pickup section 5, and a wireless transmission section 6, respectively.

Note that the arrangement state of the illumination section 4, the image pickup section 5, and the wireless transmission section 6 in FIG. 5 is schematically described for the sake of simplicity and, in reality, the arrangement state is made up as shown in FIG. 1.

Next, specific configurations of the reception antenna 12 of the capsule endoscope 2 of FIG. 5 will be described by using FIG. 6.

As shown in FIG. 6, the reception antenna 12 of the capsule endoscope 2 is configured to include a secondary side coil 2A, a secondary side core 2B, and a secondary side capacitor which is not shown.

The secondary side coil 2A, which has, for example, a substantially tubular, solenoid coil shape, is found such that the secondary side core 2B can be inserted into the inside thereof. Further, the secondary side core 2B is configured to be a cylindrical shape by using, for example, a magnetic material.

According to the configuration of the capsule endoscope 2 described above, when the node N2 as the output terminal of the frequency division circuit 17 becomes an L (Low) level based on the timing at which the node N1 as the output terminal of the magnetic field detection section 8 becomes an H (High) level, the P-channel type FET 19 is turned on so that driving power is supplied to the illumination section 4, the image pickup section 5, and the wireless transmission section 6.

That is, when an alternating magnetic field is generated by the magnetic field generation section 3, the generation of magnetic field is detected by the magnetic field detection section 8 of the capsule endoscope 2 and, based on the detection result, it becomes possible to control the power supply control section (the power supply section 7, and specifically the frequency division circuit 17 and the P-channel type FET 19 etc.) which controls the supply of driving power to the in vivo information acquisition section (the illumination section 4, the image pickup section 5, and the wireless transmission section 6, etc.).

Next, the action of the living body observation system 1 in the present embodiment will be described using FIGS. 4 and 5, and FIGS. 7A to 7D.

Note that FIGS. 7A to 7D are waveform diagrams to show the operation waveform of each principal part of FIGS. 4 and 5. FIG. 7A shows the generation state of alternating magnetic field from the magnetic field generation section 3. FIG. 7B shows the signal output (node N1) of the magnetic field detection section 8. FIG. 7C shows the signal output (node N2) of the frequency division circuit 17, which is inputted to the gate of the P-channel type FET 19 of the power supply section 7. FIG. 7D shows an operation state of the capsule endoscope 2.

A time period T1 from time t0 to time t1 shown in FIGS. 7A to 7D shows a state in which the capsule endoscope 2 is not set in the magnetic field generation section 3. Note that the setting of capsule endoscope 2 in the magnetic field generation section 3 means a state in which the capsule endoscope 2 is inserted into the primary side coil 3A of the magnetic field generation section 3 (see FIG. 4).

Suppose that at time t1 shown in FIGS. 7A to 7D, the operation to turn on a switch, which is not shown, of the magnetic field generation section 3 is performed by a user to drive the transmission antenna 11 of the magnetic field generation section 3 thereby generating an alternating magnetic field.

Then, upon generation of the alternating magnetic field, an alternating voltage is generated through electromagnetic induction at both ends of the secondary side coil 2A of the capsule endoscope 2. This alternating voltage is transformed into a direct-current voltage by the rectification section 15 which is made up of the diode 13 and the smoothening capacitor 14, and the transformed direct-current voltage, that is, the potential of the signal of the node N1, becomes an H level (V1) as shown in FIG. 7B.

Further, upon stopping of the generation of the alternating magnetic field from the magnetic field generation section 3 at time t2, the electric charge charged in the smoothening capacitor 14 is discharged via the resistor 16 and the potential of the signal of the node N1 becomes an L level.

Hereafter, in the same manner, during the time period T1 in which an alternating magnetic field is generated from the magnetic field generation section 3, the potential of the signal (detection signal) of the node N1, which is the output of the magnetic field detection section 8, becomes an H level, while during a time period T2 in which an alternating magnetic field is not generated, the potential of the signal (detection signal) of the node N1, which is the output of the magnetic field detection section 8, becomes an L level.

In the power supply section 7 to which the detection signal of the node N1, which is the output of the magnetic field detection section 8, is inputted, the signal of the node N2, which is the output of the frequency division circuit 17, acts to invert the node N2 from the previous state according to the detection signal of the node N1, which is the output of the magnetic field detection section 8, as shown in FIG. 7C.

That is, the signal of the node N2 which is the output of the frequency division circuit 17 becomes an L level during a time period T3 from time t1 to time t3, and an H level during a time period T4 from time t3 to time t5. Therefore, the P-channel type FET 19, to which gate the output (signal of the node N2) of the frequency division circuit 17 is inputted, turns into an ON state during the time period T3 from time t1 to time t3, and an OFF state during the time period T4 from time t3 to time t5.

Accordingly, during the time period T3 from time t1 to time t3, driving power from the power supply section 18 is supplied to each circuit (the illumination section 4, the image pickup section 5, and the wireless transmission section 6) of the capsule endoscope 2 and, during the time period T4 from time t3 to time t5, the supply of driving power is stopped.

That is, every time when an alternating magnetic field is generated from the magnetic field generation section 3, the starting and stopping of power supply are switched, thereby enabling a switching control of the driving state of the capsule endoscope 2 either from a deactivated state to an activated state, or from an activated state to a deactivated state.

That is, in the living body observation system 1, the alternating magnetic field to be generated makes up a kind of switching function which controls the switching of the driving state of the capsule endoscope 2.

The actions in later time periods work in the same manner as in the time periods T3 and T4.

Further, in the present embodiment, while an alternating magnetic field is generated by the magnetic field generation section 3, that is, while driving power is supplied to each circuit of the capsule endoscope 2, the magnetic flux of the alternating magnetic field emitted from the transmission antenna 11 of the magnetic field generation section 3 concentrates to the yoke 3B as described in FIG. 4. As the result of that, a major portion of the magnetic flux of the alternating magnetic field emitted from the transmission antenna 11 becomes a magnetic flux in yoke 11d, while the leakage magnetic flux 11e becomes very scarce.

As a result, since the leakage magnetic flux 11e which may cause the malfunctions of electronic equipment in the surroundings becomes very scarce, it is possible to prevent the electric equipment in the surroundings from malfunctioning. That is, it becomes possible to dispose other electronic equipment, which is necessary during examination, in the proximity of the magnetic field generation section 3, thereby improving the diagnostic performance.

Note that, in the present embodiment, the yoke 3B making up the transmission antenna 11 may be made up of a ferromagnetic material such as, for example, a ferrite, an amorphous magnetic material, and a permalloy.

Moreover, although description has been made on the case in which the secondary side core 2B making up the reception antenna 12 of the capsule endoscope 2 has an cylindrical shape, this is not limiting and the secondary side core 2B may be formed into a polygonal column shape such as, for example, a circular column shape, a triangular column shape, and a rectangular column shape. That is, the shape is not limiting provided that it allows the concentration of magnetic flux.

Further, although description has been made on the configuration in which the reception antenna 12 is provided with a secondary side core 2B, the reception antenna 12 will not be limited to such configuration and may be made up without the secondary side core 2B.

Therefore, according to the first embodiment, it is possible to realize a living body observation system 1 which can perform the control of activation and deactivation of the capsule endoscope 2 as an in vivo observation apparatus in a non-contact and low-power-consumption manner, and can maintain the deactivated state of the capsule endoscope 2 as an in vivo observation apparatus even without placing a magnet in the proximity thereof.

Moreover, since the magnetic field generation section 3 of the living body observation system 1 can reduce the leakage magnetic flux 11e included in the magnetic flux of generated alternating magnetic field to a very small amount, it becomes possible to prevent electronic equipment in the surroundings from malfunctioning. That is, it becomes possible to realize a living body observation system 1 which allows to dispose other electronic equipment, which is necessary during examination, in the proximity of the magnetic field generation section 3, thereby improving the diagnostic performance.

Note that although the first embodiment has been described by using the capsule endoscope 2 as the in vivo observation apparatus, the capsule endoscope 2 is not limiting and, needless to say, the present invention may also be applied to, for example, in vivo observation apparatuses for acquiring in vivo information such as temperature and pH levels inside the body.

### Second Embodiment

FIG. 8 is a sectional view of the magnetic field generation section of a living body observation system relating a second embodiment of the living body observation system of the present invention.

Note that among each component shown in FIG. 8, components similar to those of the living body observation system of the first embodiment are given the same reference characters omitting the description thereof, and only different parts will be described.

The living body observation system 1 of the second embodiment, although which is configured in substantially the same manner with the living body observation system 1 of the first embodiment, differs in the configuration of the transmission antenna 11 of the magnetic field generation section 3.

As shown in FIG. 8, the magnetic field generation section 3 in the second embodiment includes a transmission antenna 11A. The transmission antenna 11A is configured to include a primary side coil 3A, a yoke 3B which is disposed in the outer periphery of the primary side coil 3A, an auxiliary yoke 3C which is arranged in the bottom face of the yoke 3B, and a primary side capacitor which is not shown.

The primary side coil 3A and the yoke 3B are configured in substantially the same manner with the first embodiment.

Moreover, the newly provided auxiliary yoke 3C is configured to be a circular shape by using for example a ferromagnetic material. With this auxiliary yoke 3C being provided in the bottom face of the yoke 3B, the configuration becomes such that the opening of bottom-face side of the yoke 3B which has a cylindrical shape is closed.

Note that the auxiliary yoke 3C may be made up of a ferromagnetic material such as, for example, a ferrite, an amorphous magnetic material, and a permalloy, etc. Moreover, in the second embodiment, the yoke 3B and the auxiliary yoke 3C may be made up of the same or different materials provided that they are a ferromagnetic material. Further, in the description of the second embodiment, the yoke 3B and the auxiliary yoke 3C are separately formed, they may also be integrally formed.

Further, the auxiliary yoke 3C is not limited to having a circular shape, and may be configured to have another shape. Furthermore, the auxiliary yoke 3C may be internally attached to the inner peripheral face of the bottom portion of the yoke 3B. Further, the auxiliary yoke 3C may be configured so as to be larger than the outer diameter of the yoke 3C so that the yoke 3B is arranged on the surface of the auxiliary yoke 3C.

Other configurations are the same as those of the first embodiment.

Next, the action of the transmission antenna 11A, which is a characteristic feature of the second embodiment, will be described using FIG. 8.

In the transmission antenna 11A in the second embodiment, when an alternating magnetic field is generated, a major portion of the magnetic flux in coil 11a generated from the primary side coil 3B will pass through the auxiliary yoke 3C after passing through the yoke 3B.

That is, since a closed magnetic path is formed by the yoke 3B and the auxiliary yoke 3C, the leakage magnetic flux 11e becomes very scarce with comparison to the first embodiment.

Therefore, the transmission antenna 11A of such configuration is expected to exert a further effect of preventing the malfunction of electronic equipment in the surroundings. As the result of that, it becomes possible to dispose other electronic equipment, which is necessary during examination, in the proximity of the magnetic field generation means, thereby enabling to improve the diagnostic performance.

Thus, according to the second embodiment, since as the result of providing the transmission antenna 11A added with the auxiliary yoke 3C, the leakage magnetic flux 11e can be reduced less than that of the first embodiment, it becomes possible to further improve the effect of preventing the malfunction of electronic equipment in the surroundings caused by the leakage magnetic flux 11e. Other effects are the same as those of the first embodiment.

### Third Embodiment

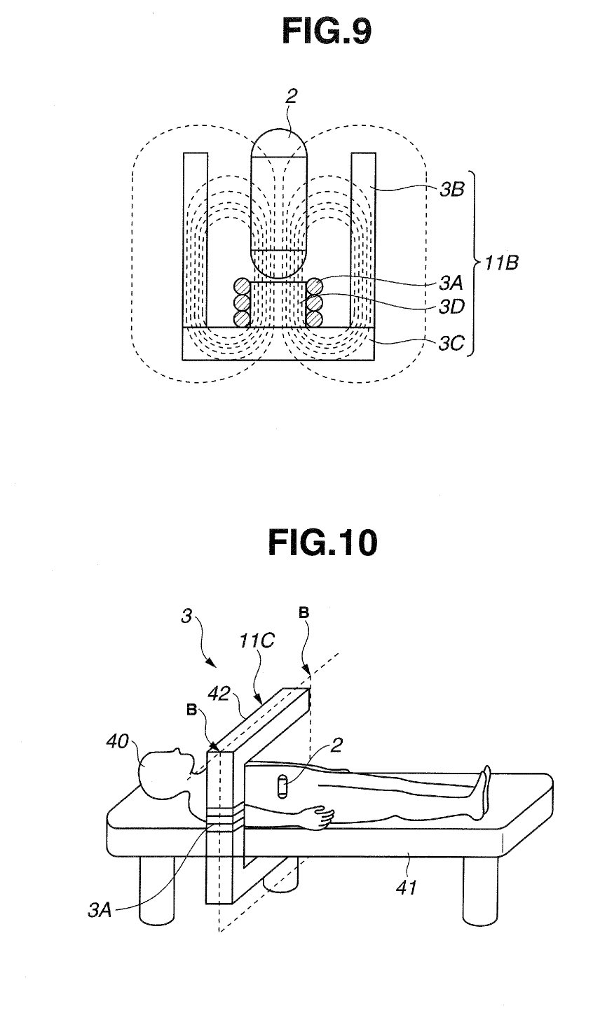

FIG. 9 is a sectional view of a magnetic field generation section of a living body observation system relating a third embodiment of the living body observation system of the present invention.

Note that among each component shown in FIG. 9, components similar to those of the living body observation system of the first embodiment are given the same reference characters omitting the description thereof, and only different parts will be described.

In the living body observation system 1 of the third embodiment, an improvement is made in the configuration of the transmission antenna 11A of the magnetic field generation section 3 of the second embodiment.

As shown in FIG. 9, the magnetic field generation section 3 in the third embodiment includes a transmission antenna 11B.

The transmission antenna 11B is configured to include a primary side coil 3A, a yoke 3B which is arranged in the outer periphery of the primary side coil 3A, an auxiliary yoke 3C which is arranged in the bottom face of the yoke 3B, a primary side core 3D which is arranged on the upper surface of the auxiliary yoke 3C and inside the primary side coil 3A, and a primary side capacitor which is not shown.

The yoke 3B and the auxiliary yoke 3C, although configurations of which are substantially the same as those of the second embodiment, are formed to have smaller sizes than those of the second embodiment. As a matter of course, the sizes thereof are large enough to provide a space into which the capsule endoscope 2 can be inserted.

Further, as shown in FIG. 9, the primary side coil 3A, although which is configured to be a substantially tubular, solenoid coil shape in substantially the same manner with the second embodiment, is formed to have smaller sizes in outer diameter and height.

Further, the primary side core 3D, which is disposed inside the primary side coil 3A, is configured to have a circular column shape by using, for example, a ferromagnetic material.

Other configurations are the same as those of the second embodiment.

Next, the action of the transmission antenna 11B, which is a characteristic feature of the third embodiment, will be described using FIG. 9.

Since, in the transmission antenna 11B in the third embodiment, the primary side core 3D is disposed inside the primary side coil 3A, it is possible to increase the self-inductance of the primary side coil 3A.

That is, since the magnetic flux to be generated when a unit current is applied to the primary side coil 3A can be increased, it is possible to improve the magnetic flux generation capability of the primary side coil 3A.

Accordingly, since not only the primary side coil 3A, but also the yoke 3B and the auxiliary yoke 3C can be reduced in size, it is possible to reduce the size of the transmission antenna 11B itself.

Note that in the third embodiment, the yoke 3B, the auxiliary yoke 3C, and the primary side core 3D may be made up of the same or different materials provided that they are a ferromagnetic material.

Further, although in the third embodiment, description has been made on the configuration in which the yoke 3B, the auxiliary yoke 3C, and the primary side core 3D are separately formed, this is not limiting and they may be integrated to form the transmission antenna 11B.

For example, configuration may be such that the yoke 3B and the auxiliary yoke 3C are integrally formed and the primary side core 3D is separately formed, or the auxiliary yoke 3C and the primary side core 3D are integrally formed and the yoke 3B is separately formed, or the yoke 3B, the auxiliary yoke 3C, and the primary side core 3D are integrally formed.

Thus, according to the third embodiment, in addition to that the effects of the second embodiment can be achieved, it becomes possible to reduce the size of the transmission antenna 111B, thereby significantly contributing to the reduction in the size of the magnetic field generation section 3 itself. Other effects are the same as those of the first embodiment.

### Fourth Embodiment

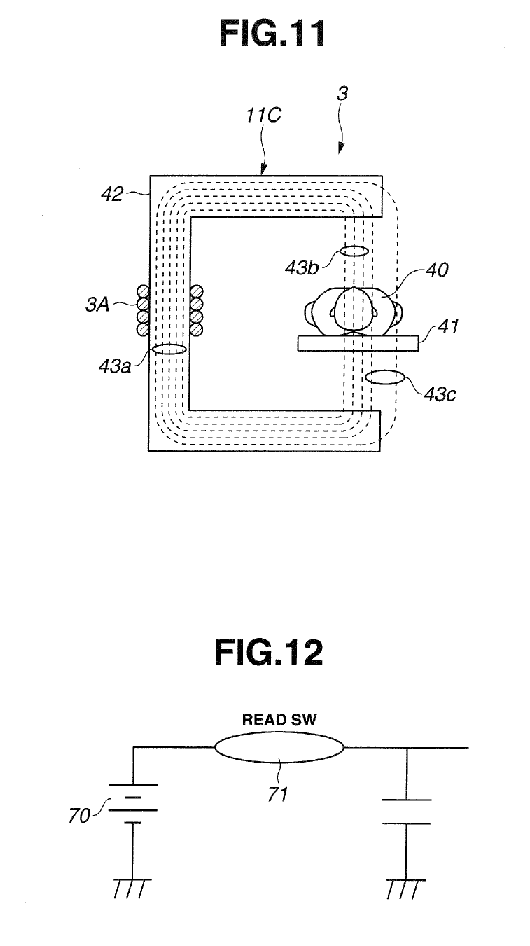

FIGS. 10 and 11 relate to a fourth embodiment of the living body observation system of the present invention. FIG. 10 is a configuration diagram to show a configuration of a magnetic field generation section of the living body observation system relating to the fourth embodiment. FIG. 11 is sectional view taken along line B-B of FIG. 10.

Note that among each component shown in FIGS. 10 and 11, components similar to those of the living body observation system of the first embodiment are given the same reference characters, omitting the description thereof, and only different parts will be described.

The living body observation system 1 of the fourth embodiment, although which has substantially the same configuration as that of the first embodiment, differs in the configuration of the magnetic field generation section 3.

Note that in the fourth embodiment, description will be made on a driving method of the capsule endoscope 2, by which the capsule endoscope 2 is activated by being applied with an alternating magnetic field from the magnetic field generation section 3 which is located in the outside, when the capsule endoscope 2 has been swallowed by a subject and has reached a desired site; and the configurations of the magnetic field generation section 3 and the transmission antenna 11 for implementing the driving method.

As shown in FIG. 10, the living body observation system 1 of the fourth embodiment includes a transmission antenna 11C which makes up a magnetic field generation section 3.

The transmission antenna 11C is configured to include a primary side coil 3A, and a core 42 which is formed into, for example, a U-shape for winding the primary side coil 3A therearound. The core 42 is to be arranged at a predetermined position of a bed 41 used for the examination of a subject 40.

Note that the number of windings of the primary side coil 3A for the core 42 is, not limited to the number of windings shown in FIG. 10, any number of windings may be used provided it enables to generate an alternating magnetic field.

Next, the method of driving the living body observation system 1 of such configuration will be described using FIGS. 10 and 11.

First, the subject 40 swallows a capsule endoscope 2 which is in a deactivated state. Thereafter, the capsule endoscope 2 is moved to a desired position in the body cavity by a peristaltic movement or a guiding system (not shown).

At that time, an operator (not shown), which is the user, drives the transmission antenna 11C shown in FIG. 10 by turning on a switch, which is not shown, of the magnetic field generation section 3 to generate an alternating magnetic field.

Then, due to the alternating magnetic field, the capsule endoscope 2 starts to be activated. For that reason, it is possible to prevent the battery from being exhausted before the capsule endoscope 2 moves to a desired position.

Further, when an alternating magnetic field is generated by the magnetic field generation section 3, as shown in FIG. 11, a magnetic flux in coil 43a generated from the primary side coil 3A passes through a core 42 and thereafter passes through the bed 41 on which the subject 40 is lying.

At this moment, the magnetic flux in coil 43a is detected by the magnetic field detection section 8 of the capsule endoscope 2 and is divided into an effective magnetic flux 43b which contributes to the activation of the capsule endoscope 2, and a magnetic flux which is not detected by the magnetic field detection section 8 and does not contribute to the activation of the capsule endoscope 2, that is, an ineffective magnetic flux 43c.

In the present embodiment, since a closed magnetic path is formed by the core 42, the ineffective magnetic flux 43c becomes very scarce, while a major portion of the magnetic flux becomes the effective magnetic flux 43b. That is, it is possible to improve the reception efficiency of alternating magnetic field thereby enabling to reduce the power consumption of the magnetic field generation section 3.

Note that although the fourth embodiment has been described on the case in which the core 42 is configured to be a U-shape, this is not limiting, and the core 42 may be, needless to say, of any shape provided that it can form a closed magnetic path.

Further, although description has been made on the configuration in which with the subject 40 lying on the bed 41 as the target, the capsule endoscope 2 is activated and deactivated from outside the subject's body, this is not limiting and, for example, the configuration may be such that with the subject 40 standing or sitting as the target, the capsule endoscope 2 is activated and deactivated from outside the subject's body.

Other configurations and actions are the same as those of the first embodiment.

Thus, according to the fourth embodiment, it becomes possible to realize: a living body observation system which enables to easily control the activation and deactivation of the capsule endoscope from outside the body in a very simple manner, to reduce the power consumption of the magnetic field generation section 3, and to restrict the exhaustion of the power supply section 18 of the capsule endoscope 2 to a minimum; and a method of driving the living body observation system. Other effects are the same as those of the first embodiment.

Note that although the fourth embodiment has been described by using the capsule endoscope 2 as the in vivo observation apparatus, the capsule endoscope 2 is not limiting, and it goes without saying that the present invention may also be applied to, for example, in vivo observation apparatuses for acquiring in vivo information such as temperature and pH levels inside the body.

The present invention can be implemented, without being limited to the embodiments and variations described above, by making various modifications within the range not departing from the spirit of the present invention.

What is claimed is:

1. A living body observation system, comprising:
an in vivo observation apparatus including: an in vivo information acquisition section for acquiring information in a living body; a power supply section for supplying driving power of the in vivo information acquisition section; a magnetic field detection section for detecting an alternating magnetic field from outside and outputting a detection result as an electric signal; and a power supply control section for controlling a supply state of driving power supplied from the power supply section to the in vivo information acquisition section, based on the electric signal; and
a magnetic field generation section which is disposed outside the in vivo observation apparatus and generates the alternating magnetic field, the magnetic field generation section including: a transmission antenna which includes a transmission coil which generates the alternating magnetic field for controlling activation and deactivation of the in vivo observation apparatus and a ferromagnetic material which is arranged in an outer periphery of the transmission coil and decreases a leakage of the alternating magnetic field in surroundings, and transmits the alternating magnetic field; a driver for driving the transmission antenna; and a power supply for supplying power to the driver.

2. The living body observation system according to claim 1, wherein
the transmission antenna of the magnetic field generating section is configured to include a core made up of a ferromagnetic material, and a transmission coil which is wound around at least a portion of the core.

3. The living body observation system according to claim 1, wherein
the magnetic field detection section is configured to include a reception coil for detecting at least the alternating magnetic field, and a reception antenna which is disposed inside the reception coil and includes a ferromagnetic material for improving a detection sensitivity of the alternating magnetic field.

4. The living body observation system according to claim 1, wherein
the in vivo observation apparatus is a capsule endoscope.

5. A driving method for driving the living body observation system according to claim 1, wherein
every time the alternating magnetic field from the magnetic field generation section is detected, the in vivo observation apparatus repeatedly gets activated and deactivated.

6. The driving method according to claim 5, wherein
the in vivo observation apparatus is swallowed into the living body of a subject, after detecting the alternating magnetic field and getting activated outside the body.

7. The driving method according to claim 5, wherein
after being swallowed into the living body of a subject, the in vivo observation apparatus is activated or deactivated by generating the alternating magnetic field outside the body.

8. The driving method according to claim 5, wherein
the in vivo observation apparatus is a capsule endoscope.
